(19) United States
(12) Patent Application Publication
(10) Pub. No.: US 2013/0059813 A1
(43) Pub. Date: Mar. 7, 2013

(54) TREATMENT OF DERMATOLOGICAL CONDITIONS

(71) Applicant: Dav Devlin, Corinth, TX (US)

(72) Inventor: Kevin P. Eaton, Dallas, TX (US)

(73) Assignee: Dan Devlin, Corinth, TX (US)

(21) Appl. No.: 13/666,778

(22) Filed: Nov. 1, 2012

Related U.S. Application Data
(63) Continuation of application No. 11/145,716, filed on Jun. 6, 2005.

(51) Int. Cl. A61K 31/714; A61P 17/06

(52) U.S. Cl. 514/52

(57) ABSTRACT

The present disclosure provides a method for treating dermatological conditions, such as psoriasis, dermatitis, and dandruff. Said conditions are treated by administering to a person a vitamin supplement composition comprising folic acid, vitamin B₁₂ and/or vitamin B₆. The vitamin supplement composition may also be essentially free of antioxidants

### CROSS-REFERENCE TO RELATED APPLICATIONS

This application claims the benefit of the filing date of U.S. Provisional Patent Application No. 60/577,294 titled “Treatment of Dermatological Conditions Such as Psoriasis” filed on Jun. 4, 2004, and U.S. Non-Provisional patent application Ser. No. 11/145,716 titled “Treatment of Dermatological Conditions” filed on Jun. 6, 2005, the disclosures of which are hereby incorporated by reference herein in their entirety.

### TECHNICAL FIELD

The present disclosure relates generally to the treatment of dermatological conditions, including but not limited to psoriasis, dermatitis, and dandruff. More specifically, the disclosure relates to the treatment of said dermatological conditions using a multiple vitamin supplement composition.

Dermatological conditions such as psoriasis, dermatitis, and dandruff affect millions of people every year in the United States alone. Psoriasis typically is characterized by dry, red patches of skin covered with silvery scales; sometimes pustules appear on top of said patches. Depending on the type of dermatitis, the following symptoms may occur: redness and itching, accompanied in severe cases by blisters and weeping sores; thickened, brownish skin; itchy, thickened, scaly skin; greasy, scaling areas at the sides of an individual's nose, between the eyebrows, behind the ears or over the breastbone; discolored (red or brown), thick and itchy skin around the shins and ankles, occasionally developing open sores or ulcers; skin around the mouth exhibiting small red bumps, pus-filled bumps or mild peeling. Signs of dandruff, of course, are white, oily-looking flakes of dead skin that dot an individual's hair and shoulders and an itchy, scaling scalp. These dermatological conditions plainly have a significant negative impact on the public appearance of afflicted individuals.

Several methods exist in the prior art for treating these dermatological conditions. Dandruff, for example is commonly treated using medicated shampoos. Depending on the type of dermatitis, creams, lotions, ointments, or oral medications are prescribed. Psoriasis, a persistent, chronic disease, also has been treated using creams, ointments, and oral medications, as well as phototherapy and biologic drugs. All of the existing treatments vary in effectiveness and their drawbacks. For instance, while existing oral medications may effectively treat even severe cases of psoriasis, significant negative side effects often result, including eye and lip inflammation, bone spurs, hair loss, liver and kidney toxicities, and birth defects if taken during pregnancy.

Therefore, it would be desirable to have a way of effectively treating dermatological conditions such as psoriasis, dermatitis, and dandruff.

### SUMMARY OF THE DISCLOSURE

The present disclosure is a treatment for dermatological conditions such as psoriasis, dermatitis, and dandruff using a vitamin supplement composition comprising folic acid, vitamin B₁₂ and/or vitamin B₆. The vitamin supplement composition may also be essentially free of antioxidants.

By “essentially free” it is meant that the vitamin composition should not contain an amount of antioxidants which would tend to damage and inactivate some of the vitamin B₁₂ and/or folic acid of the vitamin supplement. The presence of lower amounts of antioxidants would not render the vitamin composition of the present disclosure ineffective or of reduced effectiveness.

In accordance with an aspect of the present disclosure there is a provided a method of administering a multiple vitamin supplement composition for the treatment of dermatological conditions such as psoriasis, dermatitis and dandruff.

### DETAILED DESCRIPTION OF THE DISCLOSURE

Therapeutic treatment of dermatological conditions such as psoriasis, dermatitis, or dandruff with the vitamin supplement composition of the present disclosure may involve administration to persons prophylactically. That is, to prevent, retard or reduce the severity of future occurrence of such dermatological conditions.

The multiple vitamin supplement composition of the present disclosure contains a therapeutically effective amount of folic acid, vitamin B₁₂ and may or may not also contain vitamin B₆. The composition may also be essentially free of antioxidants. The vitamin composition may be administered with a pharmaceutically acceptable carrier. A pharmaceutically acceptable carrier may be any compatible, non-toxic substance suitable to deliver the components. The supplement may contain other pharmaceutically acceptable substances as required to approximate physiological conditions such as a pH adjusting and buffering agent, disbursing agents, toxicity adjusting agents, flavoring agents and like. The concentration of the components in these formulations may vary and will be selected primarily on the particular dosage and mode of administration selected. Methods for preparing supplements are well-known or will be apparent to those skilled in the art and are described in more detail in, for example, Remington's Pharmaceutical Science, 15th ed., Mack Publishing Company, Easton, Pa.

The vitamin supplement composition is useful for oral administration. Indeed, the supplement is preferably administered orally. For oral administration, solid or fluid dosage forms can be prepared. For preparing solid compositions such as tablets, the components are mixed with conventional ingredients, such as talc, magnesium stearate, and functionally similar materials, as pharmaceutical carriers. Capsules are prepared by mixing the components with an inert pharmaceutical diluent and filling the mixture into a hard gelatin capsule. Soft gelatin capsules are prepared by machine encapsulation of a slurry of the components with an acceptable vegetable oil, light liquid petrolatum or other inert oil. Fluid unit dosage forms for oil administration such as serum and suspensions can be prepared. The components may be dissolved in an aqueous vehicle together with sugar, sweetening and flavoring agents and preservatives to form a serum. Suspensions can be prepared with an aqueous vehicle and a disbursing agent such as acacia, tragacanth, methylcellulose and the like.

In the case of a vitamin supplement compound that is essentially free of antioxidants, among the antioxidants especially to be avoided is added vitamin C, and no antioxidants of any kind should be added to any of the compounds disclosed herein (although such antioxidants may be present during the preparation of such vitamins provided that they are removed afterward, either completely or at least to a level where they have virtually no effect on the vitamin components of the present disclosure). Because antioxidants may be present in vitamin preparations useful in forming the compounds of the present disclosure, the present disclosure also relates to processes wherein the folic acid, vitamin B₁₂ and/or vitamin B₆ has been tested for the presence of antioxidant and shown to be free of antioxidant. Such testing is commonly performed by liquefying a sample of the product (i.e., the vitamin, or vitamin compound, or medicament) to be tested in a solution at stomach pH, and another sample at neutral pH, incubating for 30 minutes (the gastric half-emptying time) and then assaying the amount of vitamin, or vitamins, remaining as compared to the amount prior to incubation. Note also that in the case of a vitamin supplement compound that is meant to be essentially free of antioxidants, any carrier, filler or other substance associated with the components of the disclosure used to prepare a tablet, capsule or the like should also be essentially free of anti-oxidants.

Separate vitamin supplement compositions may be prepared with each containing only folic acid, vitamin B₁₂ or vitamin B₆. Each of these tablets may also, if necessary, be essentially free of antioxidants. In this manner, one component of folic acid, vitamin B₁₂ or vitamin B₆ can be taken alone such that a user, or their physician, may have more control over the quantity of intake of folic acid, vitamin B₁₂ or vitamin B₆, without being forced to also alter the level of intake of the others. This also allows for only folic acid, vitamin B₁₂ or vitamin B₆ to be administered when appropriate.

The vitamin supplement composition may be administered in dosages and over a period of time with a frequency and duration sufficient to yield a therapeutically effective amount, i.e., an amount sufficient to curtail the severity of or eliminate the symptoms of the dermatological condition being treated. Unit dosages effective for this use will vary, but will generally range from 25 to about 2,200 micrograms of folic acid, 25 to about 2,500 micrograms of vitamin B₁₂, and 0.5 to about 20 milligrams of vitamin B₆. Preferably, the vitamin supplement composition will have unit dosages of 800 micrograms of folic acid, 115 micrograms of vitamin B₁₂, and 10 milligrams of vitamin B₆, and will be essentially free of antioxidants

1. A method of treating psoriasis by administering to a person a vitamin supplement composition comprising at least about 25 micrograms to about 2,200 micrograms of folic acid, at least about 25 micrograms to about 2,500 micrograms of vitamin B₁₂, and at least about 0.5 milligrams to about 20 milligrams of vitamin B₆, wherein no anti-oxidants are added to said composition.

2. The method of claim 1 wherein said composition is in the form of a tablet.

3. The method of claim 1 wherein said composition comprises 800 micrograms of folic acid, 115 micrograms of vitamin B₁₂, and 10 milligrams of vitamin B₆.

4. The method of claim 3 wherein said composition is in the form of a tablet.

5. A method of treating psoriasis by administering to a person a vitamin supplement composition comprising at least about 25 micrograms to about 2,200 micrograms of folic acid, at least about 25 micrograms to about 2,500 micrograms of vitamin B₁₂, and at least about 0.5 milligrams to about 20 milligrams of vitamin B₆, wherein no anti-oxidants are added to said composition and said composition contains less than 1 mg of Vitamin C.

6. The method of claim 5 wherein said composition is in the form of a tablet.

7. The method of claim 5 wherein said composition comprises 800 micrograms of folic acid, 115 micrograms of vitamin B₁₂, and 10 milligrams of vitamin B₆.

8. The method of claim 7 wherein said composition is in the form of a tablet.

9. A method of treating psoriasis by administering to a person a vitamin supplement composition comprising at least about 25 micrograms to about 2,200 micrograms of folic acid, at least about 25 micrograms to about 2,500 micrograms of vitamin B₁₂, and at least about 0.5 milligrams to about 20 milligrams of vitamin B₆, wherein no anti-oxidants are added to said composition and said composition contains no Vitamin C.

10. The method of claim 9 wherein said composition is in the form of a tablet.

11. The method of claim 9 wherein said composition comprises 800 micrograms of folic acid, 115 micrograms of vitamin B₁₂, and 10 milligrams of vitamin B₆.

12. The method of claim 11 wherein said composition is in the form of a tablet.
